(19) United States
(12) Patent Application Publication
(10) Pub. No.: US 2008/0200424 A1
(43) Pub. Date: Aug. 21, 2008

(54) Preparation for Treating Cancer

(76) Inventors: Christine Grimm, Gailingen (DE); Klaus Ehrenberger, Wien (AT); Dietmar Thurnher, Wien (AT)

Correspondence Address: MORRISS OBRYANT COMPAGNI, P.C., 734 EAST 200 SOUTH, SALT LAKE CITY, UT, 84102, US

(21) Appl. No.: 11/579,755

(22) PCT Filed: May 5, 2005
(86) PCT No.: PCT/CH2005/000254

§ 371 Date: Feb. 27, 2008

(30) Foreign Application Priority Data
May 5, 2004 (CH) 802/04

(51) Int. Cl. A61K 31/498; A61P 35/00; A61K 31/575; A61K 31/714

(52) U.S. Cl. 514/52; 514/249; 514/171

(57) ABSTRACT

The present invention relates to a combined preparation that is effective against cancerous cells containing as the active substances at least one 1-diethylaminoethyl-3-quinoxalin-2-one derivative of formula

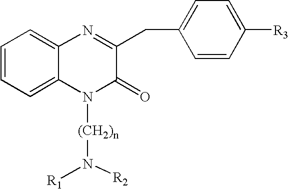

wherein R1 and R2 independently of one another are hydrogen, methyl, ethyl, propyl, butyl, or R1 and R2 together are a cycloalkyl compound;
R3 is methoxy, ethoxy, hydroxy, hydrogen, C1-C4 alkyl, halogen; and n=1, 2 or 3
or a pharmaceutically acceptable salt of said derivatives, and
an effective quantity of a cytostatic anti-cancer drug, such as a compound from the class of oxysterols or polyphenols, or a mixture containing selenium and L-cystcine and/or L-glutathione, or a mixture containing vitamins of the B-complex, in particular with the vitamins B₁ and/or B₆ and/or B₁₂, with the exception of cis- or oxaliplatin and irinotecan.

### FIELD OF THE INVENTION

The present invention relates to a preparation for treating cancer according to the pre-characterising clause of claim 1.

### PRIOR ART

EP-A-594 581 states that the delta-cholesterol-3β,7β-diol has been successfully used in the treatment of cancers of different phenotypes. The delta-cholesterol-3β,7β-diol is formed in the thymus gland and is a universal messenger substance with its own immune defense. The delta-cholesterol-3β,7β-diol owes its efficacy, which is directed only toward malignant cell surfaces, to the fact that it is non-specifically bound to LDL cholesterol, which is responsible for the transport of cholesterol into the cells. Since the receptors of LDL lipoproteins are degenerated (change in physical structure) on the surface of cancer cells, delta-cholesterol-3β,7β-diol causes blocking of the degenerated receptors. It has been found that the delta-cholesterol-3β,7β-diol is completely non-toxic and does not attack healthy cells.

U.S. Pat. No. 6,573,265 describes the use of 1-diethylaminoethyl-3-quinoxalin-2-one derivatives for the treatment of schizophrenia, paralysis of facial nerves, impaired speech following a stroke, acute hearing loss and colon cancer and skin cancer. The effect of 1-diethylaminoethyl-3-quinoxalin-2-one derivatives is attributed to their strongly anti-oxidative properties. Diseases which are caused by free radicals of oxygen metabolism can be treated in particular. According to these investigations the 1-diethylaminoethyl-3-quinoxalin-2-one derivatives are efficient radical scavengers for hydroxyl, peroxyl and peroxynitrite radicals which are responsible for a large number of diseases. U.S. Pat. No. 6,573,265 also describes the administration of caroverin (1-diethylaminoethyl-3-quinoxalin-2-one derivatives) in conjunction with conventional chemotherapy by using the preparations irinotecan (anti-neoplastic drug) and oxaliplatin to treat tumours of the colon, brain and pharynx. The effect of caroverin in specific tumours is explained in that, owing to the high anti-oxidant effect of caroverin, free radicals of the oxygen cell metabolism are eliminated and thus lasting DNA damage, which causes cancer, is prevented, i.e. it has been assumed that caroverin does not have a direct effect on cancer cells.

It is also known that 1-diethylaminoethyl-3-quinoxalin-2-one derivatives are active compounds which control an effect on the inflow and outflow of anti-cancer drugs into and out of cells (Derwent Abstract, Publication no. AN 83-45789 K). By adding the 1-diethylaminoethyl-3-quinoxalin-2-one derivatives a high concentration of the anti-cancer drug can be retained in the cells for a relatively long rime. However, it is notable that according to the above-cited publication no actual cancer-inhibiting effect is attributed to the 1-diethylaminoethyl-3-quinoxalin-2-one derivatives themselves.

Tsuro et al (Cancer Research, 42/11 (4730 to 4733 and 43, 2267 to 2272) have found that some calcium antagonists and calmodulin inhibitors can increase the intracellular concentration of vincristine and adriamycin by preventing the outflow of active substances from the cells. Calcium antagonists and calmodulin inhibitors of this type act in this case as what are referred to as modulators. According to their investigations the calcium antagonists are not directly cytotoxic for the tumour cells. This is also confirmed by another party (see Menke et al in Dtsch. med. Wschr. 113 (1986), 1728 to 1732). The efficacy of the calcium antagonists in overcoming the vincrystine or adriamycin resistance of tumour cells does not appear to be directly connected to the efficacy thereof as calmodulin inhibitors either.

WO 99/50254 relates to the compound class of quinoxalones in quite general terms and their possible use in treating different diseases. It is stated inter alia that the compound class is suitable for treating cancer.

Gerhard Maier et al (Anti-cancer Research, 19, 4251 to 4526 (1999) investigated the cytotoxicity of 7β-hydroxycholesterol and two water-soluble derivatives of 7β-hydroxycholesterol on different colon cancer cell lines. As a result of the investigations it could be proved that the 7β-hydroxycholesterols lead to cytolysis of the cancer cells.

It is also known that the uncontrolled proliferation of tumour cells can have different causes and that there are various mechanisms according to which cancer cells prevent “programmed” cytolysis of body cells. Conventional anti-cancer drugs used in chemotherapy aim at killing cancer cells. Owing to the usually limited selectivity of the chemicals used and the impossibility of having the compound act only locally, healthy body cells are also regularly affected. This means that chemotherapies of this type are associated with correspondingly strong adverse side effects.

In addition to preventing “programmed” cytolysis, for the growth of a tumour it is also necessary for the tumour cells to be adequately supplied with blood. The vascular endothelial growth factor (VEGF) plays a crucial part in this respect. The VEGF induces a neovascularisation and this allows growing tumours to be supplied with blood.

### OBJECT OF THE INVENTION

It is therefore the object of the present invention to provide improved anti-cancer drugs. In particular one aim is to provide an anti-cancer drug which attacks cancer cells at different points and/or by means of different effective mechanisms. A further aim is to provide a preparation for treating cancer that has fewer side effects.

### DESCRIPTION

According to the invention the aim is achieved by a preparation for treating cancer, containing as the active substances at least one 1-diethylaminoethyl-3-quinoxalin-2-one derivative of formula

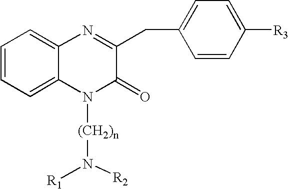

wherein R1 and R2 independently of one another are hydrogen, methyl, ethyl, propyl, butyl, or R1 and R2 together are a cycloalkyl compound;
R3 is methoxy, ethoxy, hydroxy, hydrogen, C1-C4 alkyl, halogen; and n=1, 2 or 3
or a pharmaceutically acceptable salt of said derivatives, and
an effective quantity of a cytostatic anti-cancer drug, with the exception of cisplatin, irinotecan, vincristine and/or adriamycin. To the inventors' surprise it has been found that 1-diethylaminoethyl-3-quinoxalin-2-one derivatives are capable of limiting, or even preventing altogether, the proliferation of tumour cells. Consequently it is possible to stop the growth of the tumour. In contrast to previous knowledge—diethylaminoethyl-3-quinoxalin-2-one derivatives have a direct cancer-inhibiting effect. The effect thereof is based on the fact that the supply of blood to the cancer cells may be cut off. In conjunction with cytotoxic and/or cytostatic anti-cancer drugs with apoptotic effect, an unforeseeable synergistic effect is surprisingly produced. Owing to the combination of-diethylaminoethyl-3-quinoxalin-2-one derivatives and cytotoxic anti-cancer drugs with a direct apoptotic effect, an efficient and well tolerated combined preparation is created which leads to degeneration of the tumours. The combined preparation can be used against a wide variety of tumours. To the inventors' surprise the preparation is also effective against cancerous squamous epithelial cells, combating of which is very difficult. Previous studies have shown that, surprisingly, the concentration of the cytostatic anti-cancer drug can be reduced if it is used together with diethylaminoethyl-3-quinoxalin-2-one derivatives, preferably caroverin. It is conceivable to use two or more cytotoxic and/or cytostatic anti-cancer drugs in conjunction with diethylaminoethyl-3-quinoxalin-2-one derivatives, preferably caroverin or caroverin derivatives. In particular compounds from the class of oxysterols or polyphenols or a mixture containing selenium and L-cysteine and/or L-glutathione or a mixture containing vitamins of the B complex, in particular with the vitamins B₁ and/or B₆ and/or B₁₂, are proposed as cytotoxic anti-cancer drugs combined with caroverin derivatives.

A particularly preferred preparation contains as the cytostatic anti-cancer drug at least one compound from the class of oxysterols. Oxysterols have a cytostatic effect and, surprisingly, a pronounced synergistic effect could be found in combination with the 1-diethylaminoethyl-3-quinoxalin-2-one derivatives. 7β-hydroxycholesterol or a pharmaceutically acceptable salt thereof (for example 7β-hydroxycholesterol-bis-hemisuccinate-di-sodium salt or 7β-hydroxycholesterol-bis-hemisuccinate-diethanolaminoate) is preferably used as the oxysterol. One or both hydroxyl group(s) with different substituents, in particular with mono-, bi-or tricarboxylic acids, may be substituted in this case. By suitable substitution of the 7β-hydroxycholesterol the water solubility and resorption capacity, etc. thereof may be influenced. The oxysterol used can accordingly be a compound of the following structural formulae:

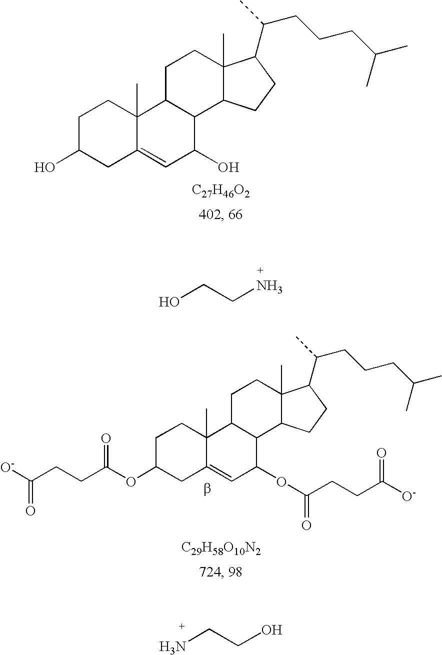

Alternatively a compound from the class of polyphenols, such as reservatol, curcumin, quercetin, gingerols or oligomeric proanthocyanidines (known by the abbreviation OPC) may be used as a cytostaticum. A further embodiment provides that an effective quantity of a mixture of selenium and L-cysteine and/or glutathione be used as the cytostaticum. In this case small quantities of selenium (1 to 50 μg) may be combined with L-cysteine and/or glutathione (10 to 100 mg). An alternative provides that an effective quantity of the vitamin B complex, in particular containing the vitamins B₁ and/or B₆ and/or B₁₂, be used in conjunction with caroverin as the anti-cancer drug.

A compound of formula

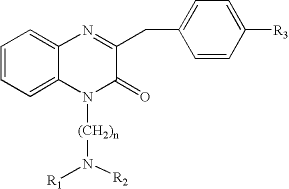

is preferably used as the 1-diethylaminoethyl-3-quinoxalin-2-one derivative, wherein R1 and R2 are an ethyl group; n=2 and R3 is a methoxy group, so the molecule is 1-diethylaminoethyl-3-(p-methoxybenzyl)-1,2-dihydro-quinoxalin-2-one (INN: caroverin) or a pharmaceutically acceptable salt. However, a derivative may also be used in which R3 is a hydroxy group, so the molecule is 1-diethylaminoethyl-3-(p-hydroxybenzyl)-1,2-dihydro-quinoxalin-2-one or a pharmaceutically acceptable salt thereof.

The present invention also relates to the use of a 1-diethylaminoethyl-3-quinoxalin-2-one derivative of formula

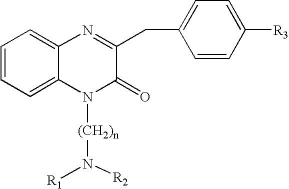

wherein R1 and R2 independently of one another are hydrogen, methyl, ethyl, propyl, butyl, or R1 and R2 together are a cycloalkyl compound;
R3 is methoxy, ethoxy, hydroxy, hydrogen, C1-C4 alkyl, halogen; and n=1, 2 or 3
or a pharmaceutically acceptable salt of said derivatives, and
an effective quantity of a cytostatic anti-cancer drug with the exception, of cis- or oxaliplatin, irinotecan. vincristine and/or adriamycin for the production of a drug for treating cancer, in particular colon cancer, carcinomas of the mucous membranes, colon cancer or breast cancer.

The invention will be described by way of example hereinafter with reference to the figures, in which:

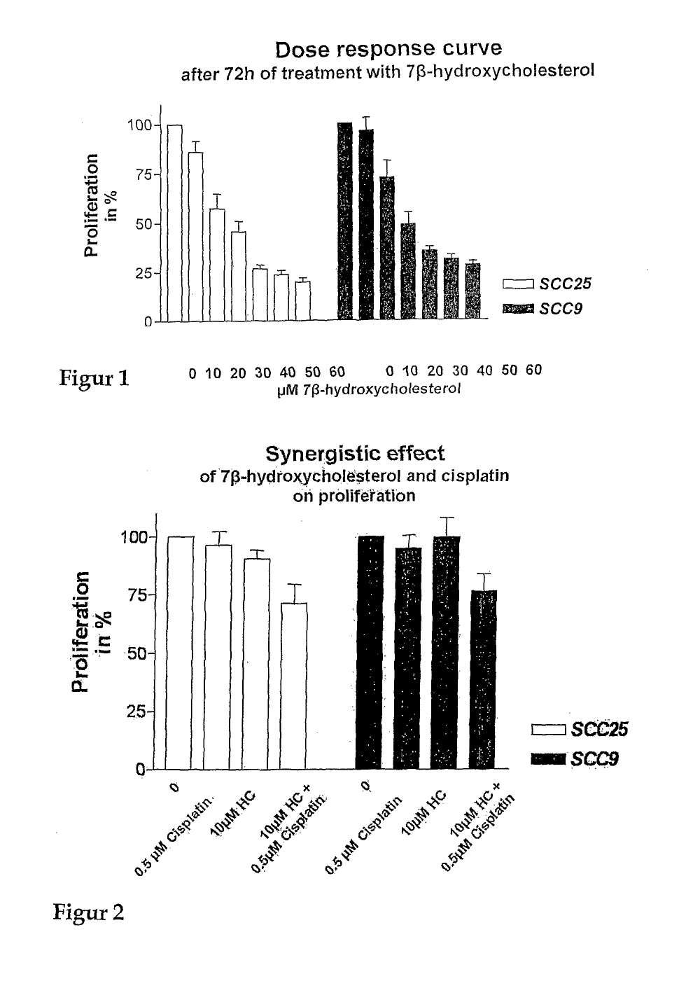

FIG. 1 shows a dose-dependent response curve of 7β-hydroxycholesterol as a function of the administered quantity of 7β-hydroxycholesterol after a treatment period of 72 hours;

FIG. 2 shows a graph of the synergistic effect of 7β-hydroxycholesterol combined with cisplatin;

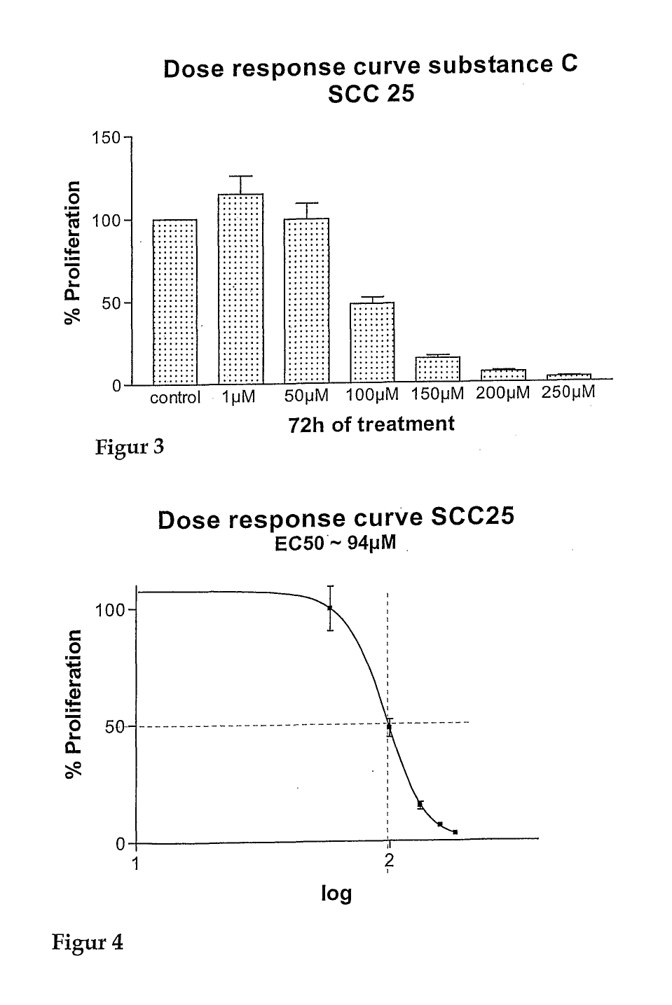

FIG. 3 shows dose-dependent response curve of caroverin against the cancer cell line SCC25;

FIG. 4 shows a response curve of caroverin (quantitative: determination of EC50 value);

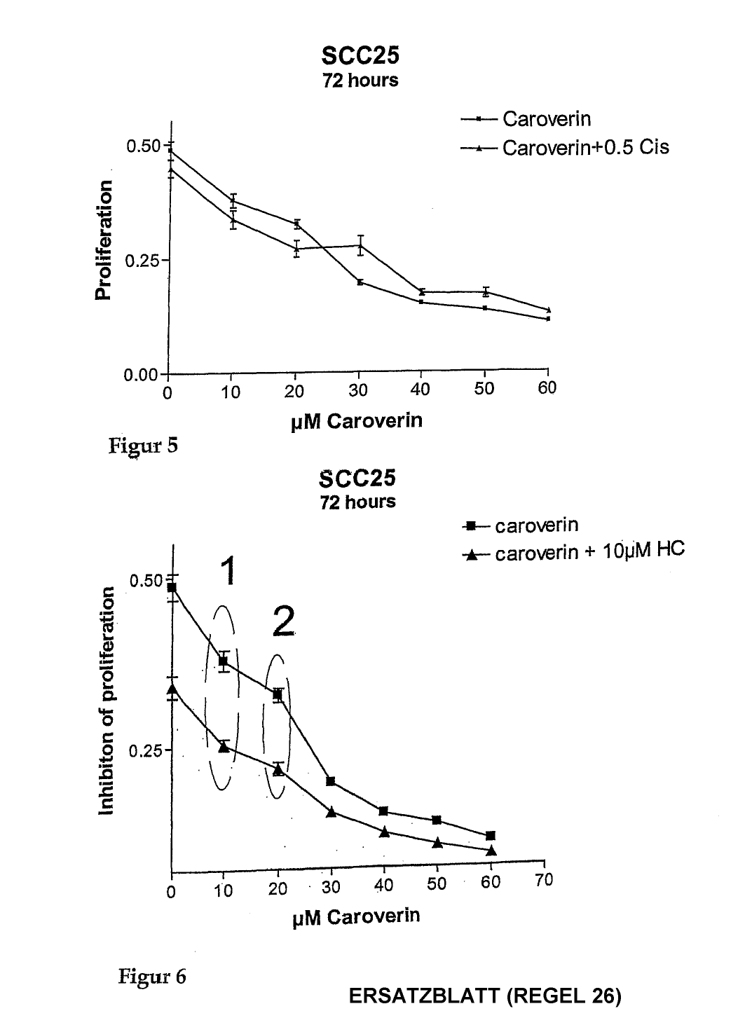

FIG. 5 shows a graph of the efficacy of caroverin combined with cisplatin;

FIG. 6 shows a graph of the synergistic effect of 7β-hydroxycholesterol and caroverin with respect to the cancer line SCC25 as a function of the concentration;

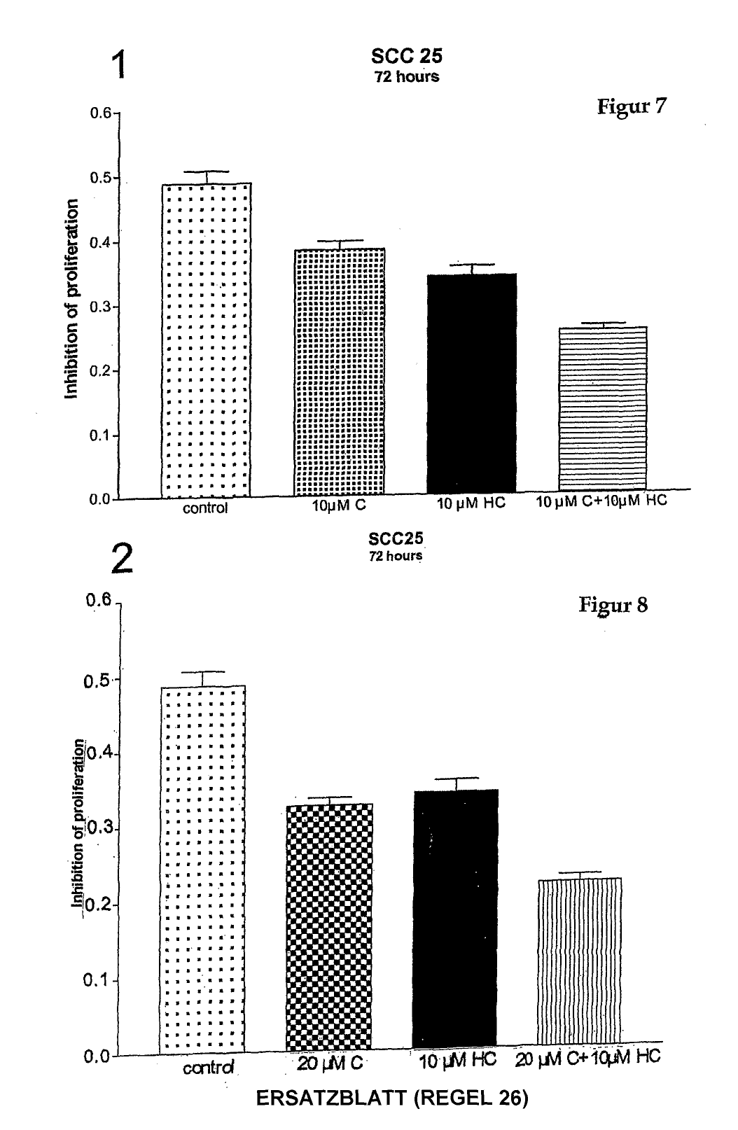

FIG. 7 shows the efficacy of 10 μm caroverin and 10 μm hydroxycholesterol alone and in combination;

FIG. 8 shows the efficacy of 20 μm caroverin and 10 μm hydroxycholesterol alone and in combination;

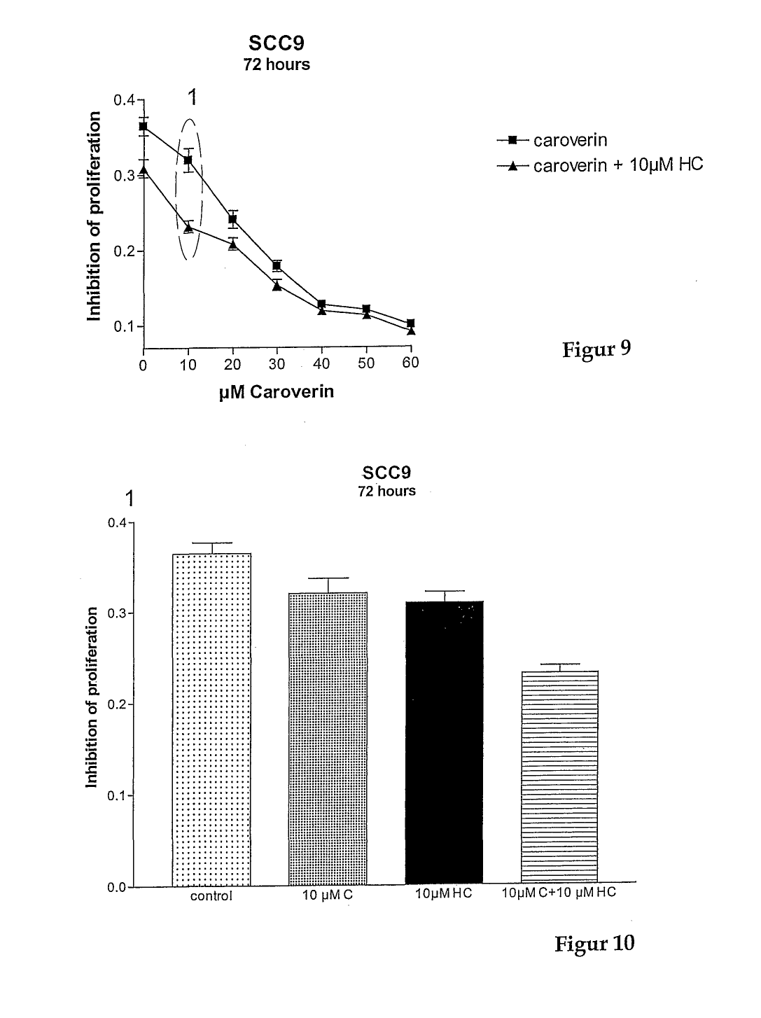

FIG. 9 shows a graph of the synergistic effect of 7β-hydroxycholesterol and caroverin with respect to the cancer cell line SCC9 as a function of the concentration;

FIG. 10 shows the efficacy of 10 μm caroverin and 10 μm hydrocholesterol alone and in combination with respect to the cancer cell line SCC9.

The biological activity of 7β-hydroxycholesterol and caroverin alone and also combined with each other was investigated. The efficacy was checked using the head and neck tumour cell lines SCC9 and SCC25. FIG. 1 shows that 7β-hydroxycholesterol (HC) is effective against both cell lines SCC9 and SCC25. The efficacy of 7β-hydroxycholesterol increases significantly as the administered quantity increases.

It can be seen in FIG. 2 that in small doses 7β-hydroxycholesterol has a synergistic effect with cisplatin.

It can be seen in FIG. 4 at which concentration the proliferation is inhibited by 50%.

A synergistic effect of caroverin in conjunction with cisplatin could not be proved, however (FIG. 5).

To the inventors' surprise it could however be proved in experiments that a combined preparation containing 7β-hydroxycholesterol and caroverin is effective against both the cancer cell lines SCC9 and SCC25 (FIG. 6 to 10). The efficacy of the combined preparation is concentration dependent. The best results are achieved at a concentration up to approximately 30 μM caroverin.

The combined preparation according to the invention can be used to treat cancerous squamous epithelial cells which occur for example in the head and neck regions. This is surprising since, as is known, squamous epithelial cells are the most difficult cancer cells to combat. Consequently it may be assumed that the preparation may also be used in the case of other, less tenacious types of cancer. Carcinomas of the mucous membranes, colon cancer and breast cancer in particular may be efficiently treated with the preparation.

The preparation can be administered intravenously, topically, orally, parenterally or transdermally. Caroverin is preferably administered in quantities of 20 to 200 mg per day, preferably between 60 and 160 mg per day. Hydroxycholesterol is preferably administered in quantities of 5 to 50 mg per day, preferably 15 to 30 mg per day. 7β-hydroxycholesterol is preferably used in neutral form (not as a salt). It has been found in clinical studies in particular that the efficacy of the combined preparation is significantly better if 7β-hydroxycholesterol is used as a neutral molecule.

The present invention relates to a combined preparation that is effective against cancer cells containing as the active substances at least one 1-diethylaminoethyl-3-quinoxalin-2-one derivative of formula

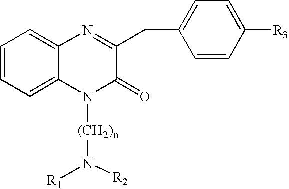

wherein R1 and R2 independently of one another are hydrogen, methyl, ethyl, propyl, butyl, or R1 and R2 together are a cycloalkyl compound;
R3 is methoxy, ethoxy, hydroxy, hydrogen, C1-C4 alkyl, halogen; and n=1, 2 or 3
or a pharmaceutically acceptable salt of said derivatives, and
an effective quantity of a cytostatic anti-cancer drug, such as a compound from the class of oxysterols or polyphenols, or a mixture containing selenium and L-cysteine and/or L-glutathione, or a mixture containing vitamins of the B-complex, in particular with the vitamins B₁ and/or B₆ and/or B₁₂, with the exception of cis- or oxaliplatin, irinotecan, vincristine and/or adriamycin.

1. A preparation for treating cancer, comprising a 1-diethylaminoethyl-3-quinoxalin-2-one derivative of formula 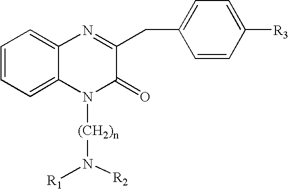
wherein R1 and R2 independently of one another are hydrogen, methyl, ethyl, propyl, butyl, or R1 and R2 together are a cycloalkyl compound;
R3 is methoxy, ethoxy, hydroxy, hydrogen, C1-C4 alkyl, halogen; and n=1, 2 or 3
or a pharmaceutically acceptable salt of said derivative, and
an effective quantity of a cytostatic anti-cancer drug, with the exception of cis- or oxaliplatin, irinotecan, vincristine, adriamycin, or a combination thereof.

2. The preparation according to claim 1, characterized in that the anti-cancer drug is an effective quantity of a compound from the class of oxysterols.

3. The preparation according to claim 1, characterized in that the anti-cancer drug is an effective quantity of a compound from the class of polyphenols.

4. The preparation according to claim 3, characterized in that one of the following compounds is used as the polyphenol: reservatol, curcumin, quercetin, gingerols, or oligomeric proanthocyanidines.

5. The preparation according to claim 2, characterized in that the oxysterol used is 7β-hydroxycholesterol or a pharmaceutically acceptable salt thereof.

6. The preparation according to claim 5, characterized in that the oxysterol used is a compound with the following structural formula:

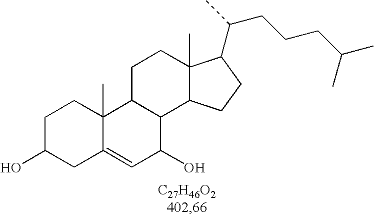

7. The preparation according to claim 5, characterized in that the oxysterol is 7-βhydroxycholesteryl-bishemisuccinate-di-ethanolamine salt with the formula

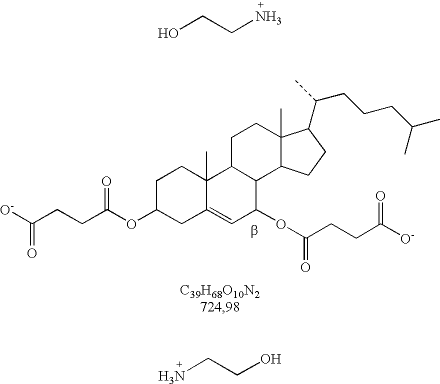

8. The preparation according to claim 1, characterized in that the anti-cancer drug is an effective quantity of a mixture of selenium and L-cysteine and/or L-glutathione.

9. The preparation according to claim 1, characterized in that the anti-cancer drug comprises an effective quantity of a vitamin B complex, containing vitamin-B₁, B₆, B₁₂, or a combination thereof.

10. The preparation according to claim 1, wherein R1 and R2 are an ethyl group; n=2 and R3 is a methoxy group, so the molecule is 1-diethylaminoethyl-3-(p-methoxybenzyl)-1,2-dihydro-quinoxalin-2-one (INN: caroverin) or a pharmaceutically acceptable salt.

11. The preparation according to claim 1, wherein R1 and R2 are an ethyl group, n=2 and R3 is a hydroxyl group, so the molecule is 1-diethylaminoethyl-3-(p-hydroxybenzyl)-1,2-dihydro-quinoxalin-2-one or a pharmaceutically acceptable salt thereof.

12. A method of treating cancer, said method comprising:
administering 1-diethylaminoethyl-3-quinoxalin-2-one derivative of formula; 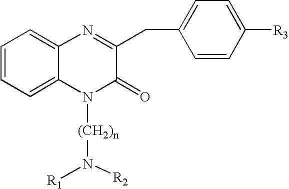
wherein R1 and R2 independently of one another are hydrogen, methyl, ethyl, propyl, butyl, or R1 and R2 together are a cycloalkyl compound;
R3 is methoxy, ethoxy, hydroxy, hydrogen, C1-C4 alkyl, halogen; and n=1, 2 or 3
or a pharmaceutically acceptable salt of said derivatives, and
an effective quantity of a cytostatic anti-cancer drug, with the exception of cis- or oxaliplatin, irinotecan, vincristine and/or adriamycin, thereby treating cancer.

13. The method according to claim 12, characterized in that the cancer to be treated is breast cancer.

14. The method according to claim 12, characterized in that the cancer to be treated is colon cancer.

15. The method according to claim 12, characterized in that the cancer to be treated is a carcinoma of the mucous membranes.

16. (canceled)

17. A pharmaceutical composition comprising a 1-diethylaminoethyl-3-quinoxalin-2-one derivative of formula: 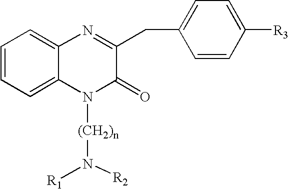
wherein R1 and R2 are independently hydrogen, methyl, ethyl, propyl, butyl, or R1 and R2 together are a cycloalkyl compound;
R3 is methoxy, ethoxy, hydroxy, hydrogen, C1-C4 alkyl, halogen; and n=1, 2 or 3
or a pharmaceutically acceptable salt of said derivative,
and an effective amount of a cytostatic anti-cancer drug selected from the group consisting of an oxysterol, polyphenol, a mixture containing selenium and L-cysteine, L-glutathione, or a combination of L-cysteine and L-glutathione, and a B-complex vitamin.

18. The pharmaceutical composition of claim 17, wherein the cytostatic anti-cancer drug is vitamin B₁, B₆, B₁₂ or a combination thereof.

19. The pharmaceutical composition of claim 17, wherein the 1-diethylaminoethyl-3-quinoxalin-2-one derivative is diethylaminoethyl-3-(p-methoxybenzyl)-1,2-dihydro-quinoxalin-2-one (INN: caroverin), 1-diethylaminoethyl-3-(p-hydroxybenzyl)-1,2-dihydro-quinoxalin-2-one, or a pharmaceutically acceptable salt of said derivative, and the cytostatic anti-cancer drug comprises 7β-hydroxycholesterol.

20. The preparation according to claim 6, wherein the 1-diethylaminoethyl-3-quinoxalin-2-one derivative is diethylaminoethyl-3-(p-methoxybenzyl)-1,2-dihydro-quinoxalin-2-one (INN: caroverin), 1-diethylaminoethyl-3-(p-hydroxybenzyl)-1,2-dihydro-quinoxalin-2-one, or a pharmaceutically acceptable salt of said derivative.

21. The preparation according to claim 7, wherein the 1-diethylaminoethyl-3-quinoxalin-2-one derivative is diethylaminoethyl-3-(p-methoxybenzyl)-1,2-dihydro-quinoxalin-2-one (INN: caroverin), 1-diethylaminoethyl-3-(p-hydroxybenzyl)-1,2-dihydro-quinoxalin-2-one, or a pharmaceutically acceptable salt of said derivative.
